(19) United States
(12) Patent Application Publication
(10) Pub. No.: US 2001/0008760 A1
(43) Pub. Date: Jul. 19, 2001

(54) REAGENT SYSTEM AND KIT FOR DETECTING HIV INFECTED CELLS

(76) Inventors: CHESTER F. KING, FREDERICK, MD (US); ROBERT A. HALLOWITZ, GAITHERSBURG, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/139,663

(22) PCT Filed: Oct. 15, 1997
(86) PCT No.: PCT/US97/18649

§ 371 Date: None

(51) Int. Cl. C12Q 1/70

(52) U.S. Cl. 435/5

(57) ABSTRACT

This invention relates to blood collection and diagnostics. More particularly, the invention relates to blood collection and diagnostics utilizing techniques such as magnetic separation and photodetection. The present invention also relates to methods and an apparatus for detecting the presence of antigens displayed on the surface of cells. More preferably, the present invention relates to the detection of cells infected by human immunodeficiency virus (HIV) and related viruses. In accordance with the present invention, HIV-infected cells can be detected and separated from uninfected cells. In a preferred embodiment, separation is achieved by a magnetic field. By coating the infected cells with magnetic particles, transfer of the cells to a precise location is facilitated. A novel aspect of the present invention is a cartridge antigen test which allows for the collection and mixing of blood with reagents in one package, which can be viewed on a fluorescent microscope.

### CROSS-REFERENCE TO RELATED APPLICATIONS

This application is a continuation-in-part of PCT/US97/18649 filed Oct. 15, 1997, which is a continuation in part of U.S. application No. 08/732,782 filed Oct. 15, 1996, and U.S. application No. 08/732,784 filed Oct. 15, 1996.

### FIELD OF INVENTION

The present invention relates to methods for detecting the presence of antigens displayed on the surface and the interior compartments of cells. More preferably, the present invention relates to the detection of cells infected by microorganisms, viruses, etc., such as human immunodeficiency virus (HIV), other retroviruses, DNA viruses, RNA viruses, and non sexually-transmitted viruses. Detection of infection is achieved earlier and more accurately than with previous methods. In accordance with the present invention, virus infected cells can be detected and separated from uninfected cells. In a preferred embodiment, separation is achieved by a magnetic field. By coating the infected cells with magnetic particles, transfer of the cells to a precise location is facilitated.

The present invention relates to methods and materials useful wherever the isolation and quantitation of infected cells is of value, such early diagnosis of HIV infection, determination of the infectivity of a specific strain or isolate of HIV-1 on a specific target cell population in tissue culture, the assessment of the ability of neutralizing antibodies or sera or drugs to decrease infectivity of said viral strain, etc. More particularly, in one embodiment, the invention provides compositions and methods for utilizing a cocktail or monoclonal or polyclonal anti-surface protein antibodies conjugated with a tag, such as FITC, and beads coated with an antibody produced against the tag, such as anti-FITC microbeads.

### BACKGROUND

The state of the art with respect to the epidemiology and immunology of the causative agent of AIDS in humans is well summarized in: Laurence, “The Immune System and AIDS,” Scientific American, December, 1985, pp. 84-93; Gallo, “The First Human Retrovirus,” Scientific American, December, 1986, pp. 88-98; Gallo, “The AIDS Virus,” Scientific American, January, 1987, pp 47-56; Levy et. al., Science, 225, 840-842 (1984); “Mobilizing against AIDS,” Institute of Medicine, National Academy of Sciences, Harvard University Press (Cambridge Mass. 1986); and Lane et. al., Ann Rev. Immunol., 3, pp.477-500 (1985).

The role of the CD4 surface glycoprotein of human T lymphocytes in infection by HIV has been extensively studied as represented by Dalgeleish et al., Science, 312, pp. 763-767 (1984); Klatzmann et al., Science, 767-768 (1984); Klatzmann et al., Science, 225, pp. 59-62 (19984); McDoual et al., J. Immunol., 135, pp.3151-3162 (1985); and Maddon et al., Cell, 47, pp.333-348 (1986).

Infection of a T-cell or macrophage or monotyte (CD4 bearing mono-nuclear cells) follows from interaction between an epitope borne by HIV-1 and the CD4 receptor which is located ultimately on the cell surface. The epitope on HIV-1 is borne by the envelope glycoprotein gp120 (molecular weight 120 kilodaltons). The glycoprotein GP 120 is structurally exposed on the outside of the HIV-1 envelope. The gp 120/gp41 binds and fuses to the target cell, permitting the injection of the viral genome into the target cell. After a complex process of decoding each strand of RNA and creating complementary DNA, which is then integrated into the host cell genome, the cell begins to produce all of the components of new viruses. The production of the envelope proteins of the viruses begins inside the cell and rapidly proceeds to the expression of gp120/41 complex intermingled with host cell membrane after leaving the endoplasmic reticulum on the target cell surface.

In light of the above background information regarding HIV and AIDS, it can be deduced that antibodies specific for the envelope of the virus, which plays such an important role in the establishment of the viral infection, could have great significance in identifying the most crucial cell-bound antigens on the surface of infected cells in the peripheral blood.

A number of research groups have reported successful development of murine monoclonal antibody specific for gp120. For example, T. C. Can et al. (Eur. J. Immunol. 16:1465, 1986) reported that they chemically synthesized a portion of the peptide chain of gp120 and then prepared monoclonal antibodies (MAbs) specific for the synthetic peptide. They employed those MAbs in an indirect fluorescent antibody technique and reported they were able to detect HIV infection with greater sensitivity than was possible with the reverse transcriptase determination technique. Additional reports of murine anti-gp 120 MAbs have been reported by Gostling et al., (J. Clin. Microbiol.: 25,845, 1987) and Matsushida et al., (Medical Immunol. 14: 307, 1987).

Testing serum for antibodies to HIV is currently the most cost-effective and accurate method of screening for and confirmation of infection. References 1-5 “AID Knowledge Base 2.1-9, Centers for Disease Control—Update: Serologic Testing for Antibody to Human Immunodeficiency Virus”, MMWR 1988; 36: 833-40; Schwartz, J. S., Dans, P. E., “Kinosian BP Human Immunodeficiency Virus Test Evaluation, Performance and Use”, JAMA 1988; 259: 2574-9; Burke, D. S., Brundage, J. F., Redfield, R. R. et al., “Measurement of the False Positive Rate in a Screening Program for Human Immunodeficiency Virus Infections”, New England Journal of Medicine, 1988; 319: 961-4; Cohen, N. D., Munoz, A., Reitz, B. A. et al. “Transmission of Retroviruses by Transfusion of Screened Blood in Patients Undergoing Cardiac Surgery”, New England Journal of Medicine 1989; 320: 1172-6; MacDonald, K. L., Jackson, J. B., Bowman, R. J. et al., “Performance Characteristics of Serologic Tests for Human Immunodeficiency Virus Type 1 (HIV-1) Antibody Among Minnesota Blood Donors. Public Health and Clinical Implications”, Ann Intern Med. 1989; 110: 617-21.

HIV antibody tests have their limitations. Usually antibodies to HIV appear within 3-6 months and as early as 6-8 weeks, but silent infections have been documented in which seroconversion has occurred as late as 3 years from the moment of exposure. Therefore, because an infected person does not develop antibodies immediately, a negative test result cannot rule out HIV infection.

It has been shown that the majority (90%) of people first testing positive for HIV will develop AIDS within one year. This strongly suggests that the average person identifying HIV infection has been positive for an average of 8-9 years, in view of the fact that the average interval between infection an AIDS is 9-10 years.

This is particularly problematic because of the behavioral studies indicating that a person practicing high risk behaviors is likely to seek testing within days or a few weeks of the high risk behavior. That person is then likely to forget about HIV and continue risky behavior based on the false reassurance of a negative test performed before seroconversion was even possible.

The consequences of the above observations are: 1. The majority of people practicing intermittent high risk behaviors tend to seek reassurances very shortly after committing such behaviors; 2. They get reassurance and false security because of false-negative tests based on as yet undetectable antibody levels; 3. The majority of infected people continue intennittent or continuous high risk behaviors for 8-9 years after becoming infected; 4. These people are, therefore, transmitting HIV for 8-9 years; 5. If affordable accurate testing could be accomplished within the brief interval between risky behavior and seroconversion, a significant increase in early HIV detection would be likely; 6. Therefore, affordable early detection would create a significant reduction in the high prevalence of HIV transmission by the falsely assured and oblivious people in the 90% majority cited above.

In a study of consistent sequential detection of RNA, antigen and antibody in early HIV infection, sequential appearance in blood of HIV RNA, HIVag, and HIV antibody was found. Data derived from testing Seroconversion Panels demonstrate a consistent sequential rise in the concentrations of HIV RNA followed by HIV antigen (p24), followed by anti-HIV in early HIV infection. Based on the timing of the appearance of RNA and antigen it was concluded that HIV RNA and HIVag could be used to confirm early infection. RNA and/or HIVag tests were concluded to be potentially useful for earlier detection of HIV infection (e.g., blood screening). Busch, M., Schumacher, Richard T., Stramer, S., et. al. “Consistent Sequential Detection of RNA, Antigen and Antibody in Early HIV Infection: Assessment of the Window Period” Irwin Memorial Blood Center, San Francisco, Calif., Boston Biomedical, Inc., Bridgewater, Mass., Poster presented at XI International AIDS Conference, Vancouver, BC July 1966.

Efforts have been made to close this “window” between exposure and antibody detect ability. The p24 antigen test has already been mandated for use by all registered blood and plasma centers because of a partial closure of the “window” achieved by this method. However, in the best-case scenario, p24 antigen detection realistically only closes the window by 6-7 days. A syndrome of primary infection has been shown to occur in the majority of HIV-1 infected patients between days 5 and 30 following exposure to and infection with the HIV-1 virus. During this interval a very intense viremia has been discovered, and as well, an outpouring of peripheral blood mononuclear cells occurs (Dr. Mark Lewis, personal communication), many of which are suggested to be productively infected. In addition animal studies using the SHIV (HIV-1 envelope with SIV core) model demonstrate that following the vaginal inoculation, SHIV is detectable in the blood stream with sensitive culture techniques within 2 days of exposure (Dr. Mark Lewis, personal communication). Since a significant rate of viral reproduction occurs within the first week of infection causing the presence of the envelope glycoprotein GP 120 bearing lymphocytes in the peripheral blood, detection of blood-bound GP 120 is an effective means to close he “window” even further than the p24 test.

A method for determining the concentration of substances in biological fluids (e.g., drugs, hormones, vitamins and enzymes) wherein magnetically responsive, permeable, solid, water insoluble, micro particles are employed is disclosed in U.S. Pat. No. 4,115,534. Functional magnetic particles formed by dissolving a mucopolysaccharide such as chitosan in acidified aqueous solution containing a mixture of ferrous chloride and ferric chloride is disclosed in U.S. Pat. No. 4,285,819. The micro spheres may be employed to remove dissolved ions from waste aqueous streams by formation of chelates. U.S. Pat. No. 3,933,997 describes a solid phase radio immunoassay for digoxin where anti-digoxin antibodies are coupled to magnetically responsive particles.

Small magnetic particles coated with an antibody layer are used in U.S. Pat. No. 3,970,518 to provide large and widely distributed surface area for sorting out and separating select organisms and cells from populations thereof. U.S. Pat. No. 4,018,886 discloses small magnetic particles used to provide large and widely distributed surface area for separating a select protein from a solution to enable detection thereof. The particles are coated with a protein that will interact specifically with the select protein.

U.S. Pat. No. 4,070,246 describes compositions comprising stable, water insoluble coatings on substrates to which biologically active proteins can be covalently coupled so that the resulting product has the biological properties of the protein and the mechanical properties of the substrate, for example, magnetic properties of a metal support.

A diagnostic method employing a mixture of normally separable protein-coated particles is discussed in U.S. Pat. No. 4,115,535. Micro spheres of acrolein homopolymers and copolymer with hydrophilic comonomers such as methacrylic acid and/or hyroxyethylmethacrylate are discussed in U.S. Pat. No. 4,413,070. U.S. Pat. No. 4,452,774 discloses magnetic iron-dextran micro spheres which can be covalently bonded to antibodies, enzymes and other biological molecules and used to label and separate cells and other biological particles and molecules by means of a magnetic field. Coated magnetizable micro particles, reversible suspensions thereof, and processes relating thereto are disclosed in U.S. Pat. No. 4,454,234. A method of separating cationic from anionic beads in mixed resin beds employing a ferromagnetic material intricately incorporated with each of the ionic beads is described in U.S. Pat. No. 4,523,996. A magnetic separation method utilizing a colloid of magnetic particles is discussed in U.S. Pat. No. 4,526,681. U.K. Patent Application GB No. 2,152,664A discloses magnetic assay reagents.

An electron-dense antibody conjugate made by the covalent bonding of an iron-dextran particle to an antibody molecule is reported by Dutton et al. (1979) Proc. Natl. Acad. Sci. 76:3392-3396. Ithakissios et al. describes the use of protein containing magnetic micro particles in radioassays in Clin. Chem. 23-2072-2079 (1977). The separation of cells labeled with immunospecific iron dextran micro spheres using high gradient magnetic chromatography is disclosed by Molday et al. (1984) FEBS, 17: 232-238. In J. Immunol. Meth. 52-353-367 (1982) Molday et al. describe an immunospecific ferro-magnetic iron-dextran reagent for the labeling and magnetic separation of cells. An application of magnetic micro spheres in labeling and separation of cells is also disclosed by Molday et al. in Nature 268.437-437 (1977). A solid phase fluoroimmunoassay of human albumin and biological fluids is discussed by Margessi et al. (1978) Clin. Chim. Acta. 89:455-460. Nye et al. (1976) Clin. Chim. Acta. 69:387-396 discloses a solid phase magnetic particle radioimmunoassay. Magnetic fluids are described by Rosenweig (1983) Scien. Amer. 10:136-194. Magnetic protein A micro spheres and their use in a method for cell separation are disclosed by Widder,et al. (1979) Clin. Immunol. and Immunopath. 14:395-400.

U.S. Pat. No. 5,279,936 is a method directed to the separation of a component of interest from other components of a mixture by causing the binding of the component of interest to magnetic particles. In the embodiment of the invention which is a method to separate cells from a mixture containing other components, the method comprises layering a first liquid medium containing cells and other components with a second medium which is of a different density than and/or different viscosity than the first liquid medium. The cells are bound to paramagnetic particles. The layered first liquid medium and the second liquid medium are subjected to a magnetic field gradient to cause the cell particles to migrate into the second medium. The purpose of isolating the cells in the second liquid medium is to then by a further embodiment to separate the cells from the second liquid medium. In the current invention, there is no need for a second liquid medium because the magnetic separation of HIV-1 infected cells is accomplished in the medium of PBS diluted blood, by bringing the infected cells to a predetermined point in the reaction vessel. The only task required after separation is the illumination of the point of highest magnetic field concentration, to ascertain the presence or absence of high density specific fluorescence, which if present would indicate the presence of fluorescently tagged HIV infected peripheral blood leucocytes (pb1).

U.S. Pat. No. 4,935,147 is a method that specifically targets the application of magnetic separation in the assay of organic and inorganic biochemical analytes, particularly those analytes of interest in the analysis of body fluids. The method of the mentioned patent provides a way of separating non-magnetic particles from a medium by virtue of the chemically controlled non-specific reversible binding of such particles to magnetic particles. Because of the small size of the magnetic particles, it also provides for a very rapid binding of a substance to be separated. By then aggregating the particles there is provided a much more rapid and complete magnetic separation than has been achieved by previous methods. In the current invention, this technique of magnetic separation does not apply because of the fact the antigen of interest is bound to cells, and therefore not in solution or in need of agglutinaton for separation. The current invention merely requires the adherence of the many magnetic particles to an infected cell surface to magnetically pull the entire cell of interest to a predetermined point in the reaction vessel for viewing.

With respect to a kit, the prior art collected blood for testing in multiple steps. The first step was to collect the blood into a suitable container from a puncture wound in the skin of a finger or by venipuncture. Then the blood would have to be placed into a container suitable for transporting or mixing with test reagents. Then reagents would have to be added in a multiple step fashion, interrupted by wash steps. The problem with this approach is multiple steps which are time consuming and require training. In the collection of blood, the prior art is still dealing with the lance and test tube methods.

For example, the aforementioned U.S. Pat. 4,777,964 to David Briggs, Kent A. Leger, Brenda Briggs (Oct. 18, 1988) provides a system for whomever wishes to ascertain whether or not he is carrying the AIDS virus to perform a blood sampling and to forward the sample to a lab for the further testing. The kit contains a lance and a tube for collecting the sample and requires the user to seal the tube at the ends with putty. This device and kit is only a means for collecting blood and keeping the sample intact for mailing to a laboratory for further testing. No tests are performed using the appliances provided. In addition, the sample must be transferred to a testing vessel and mixed with the appropriate testing medium. There are a host of other test kits and methods for collection and preparation of specimens. The following patents are of interest with respect to this field: U.S. Pat. No. 4,382,062 to Kohl (May 3, 1983); U.S. Pat. No. 4,365,970 to Lawrence et al. (Dec. 28, 1982); U.S. Pat. No. 4,122,947 to Falla Oct. 31, 1978); U.S. Pat. No. 3,272,319 to Brewer (Sep. 13, 1966); U.S. Pat. No. 3,203,540 to Kalt et. al. (Aug. 31, 1965). None of the above-mentioned patents provide sample collection, preparation and observation of the immunochemical reaction in the same vessel.

Some test media provide for the performance of the magnetic separation, but do not provide for the reaction to occur in the collection apparatus, nor can the complete test be performed outside a controlled laboratory environment where multiple steps must be performed. U.S. Pat. No. 5,186,827 to Paul A. Liberti, Brian P. Feeley, Dhanesh I. Gohel (Feb. 16, 1993) describes an elaborate magnetic separator for separating magnetic particles from a non-magnetic test medium. The magnetic separator includes a non-magnetic container having a peripheral wall with an internal surface area for receiving the test medium, and magnetic means for generating a magnetic field gradient within the container in which tested material is contained in reaction vessels such as test tubes.

There are also methods that utilize magnetic separation and the use of light sources to identify particles. U.S. Pat. No. 5,238,810 to Koichi Fujiwara, Juichi Noda, Hiroko Misutani, Hiromichi Mizutani (Aug. 23, 1993) provides for such a process; however, as with other magnetic separation methods, this method involves multiple apparatus and steps just to collect and prepare the blood samples for testing. This method also focuses on using one reagent for its test, rather than on a double reagent mixture. It provides for various vessel configurations for performing the reaction, but does not include or contemplate a vessel that has served as reagent storage, blood collection, mixture, incubation and viewing device in one.

### SUMMARY OF THE INVENTION

The present invention relates to a variety of assays for detecting and/or separating, e.g., pbls, T-cells, immortalized cell lines, macrophages, monocytes, CD4 bearing cells, artificial liposomes, etc., utilizing magnetic separation technology. In a particular aspect, it relates to obtaining a blood sample and mixing it with testing reagents in one step, and in one disposable vessel. The vessel can be incubated and the related results of reaction between the reagents and the blood sample can be viewed and read in the vessel by a fluorescent microscope without additional processing for quick and accurate testing.

The present invention is directed to blood collection and magnetic separation apparatus and methods in which antibody-coupled magnetic particles and antibody -conjugated flourochromes are used to isolate substances of interest from a non-magnetic test medium by means of high gradient magnetic separation and identification by application of focused light.

An aspect of the present invention relies upon a unique reaction vessel that serves the multiplicity of purposes as stated above. The prior methods of magnetic separation differ in various ways, e.g., because of the incompatibility of reaction vessel configuration with blood sample collection and single-step testing. In addition, most magnetic separation devices do not provide for viewing any further reaction within the vessel.

The current invention provides a self-contained micro-baggy of reagents that is punctured and permitted to mix with the sample of blood at the same time the sample is being collected. Further, the chamber in which the blood is collected, and in which the reagents are mixed with the blood, is also the same chamber or vessel used for incubating the reaction mixture, and further, is the chamber in which magnetic separation of the infected cells, if present, is performed. Finally it also serves as the chamber in which the infected cells, if present, are viewed. There is no equivalent multipurpose chamber such as the present invention that provides for blood collection reagent storage, reagent/blood mixing, reaction incubation, magnetic separation and finally, viewing of any infected cells present.

The Cartridge Antigen Test (CAT) is a device that permits blood collection, reagent mixing with blood, incubation of the mixture, magnetic separation, and viewing of the test results. The device consists of a well slide with micro-lances, a micro-baggy full of reagents, a mylar cover strip, and a bar code for identification purposes.

The present invention also relates to a fluorometric immunoassay in which a pair of manufactured non-competitive antibodies to a surface antigen, such as gp120, are utilized. One antibody (Mab) is coupled to paramagnetic particles, while the second in conjugate with FITC. The present invention takes advantage of the technology of immunomagnetic separation developed over the past 15 years to enrich or separate out of a mixture of cells, specific cellular components based on their specific immunological markers. See, e.g., U.S. Pat. Nos. 4,777,145; 4,731,337; 5,186,827; 5,238,810; 5,279,936; 5,411,863; and 4,935,147.

In these inventions particular methods are disclosed for separating a substance from a liquid medium using magnetic particles. None of these inventions, however, are specific for the process of using immunomagnetic particles for the diagnosis of HIV in whole blood. The present invention relies upon the commercial availability of high affinity anti-gp120 Mabs coupled with magnetic particles and a second non-competitive anti-gp120 Pab conjugated with FITC to fluorimetrically “tag” an HIV infected cell and then magnetically separate it from uninfected cells in whole blood.

Of particular importance to the background of the present invention is the consideration of factors that demonstrate the importance of creating a diagnostic system which takes advantage of the above described molecular biology of HIV infection. It is also important to understand the need for the present invention based upon the limitations posed by current screening and confirmatory test protocols which are still mainly dependent upon host immune response to HIV infection by antibody production.

Accordingly, several objects and advantages of our invention are the objective of placing the entire process of stabbing the finger, collecting the blood, treating the blood with test reagents, incubating the text mixture and reading the results form a single device with no transfers, additions, or complicated processes. The operator requires no special training to use the device. This allows for faster, automated testing of the results in remote sites and easy labeling of patient's tests and easy disposal of samples.

Still further objects and advantages will become apparent form a consideration of the ensuring description and accompanying drawings.

### BRIEF DESCRIPTION OF THE DRAWINGS

FIG. 1 is a frontal view of a collection/processing cartridge according to the present invention.

FIG. 2 is a side view of the collection/processing cartridge illustrating a well, microlances, a micro-baggy and a mylar cover.

FIG. 3 is an enlarged view of one of the micro-lances shown in FIG. 2.

FIG. 4A-4C are side views illustrating the collection of a blood sample from a test subject.

FIG. 5A-5E are side views of the well and illustrating an immunochemical reaction between blood and a two reagent system including incubation, application of a magnetic gradient, and the application of a focused light source on the reagent and blood mixture.

FIG. 6A-6C are top and side views respectively, of the cartridge and well illustrating incubation, the application of a magnetic gradient and a focused light source, and the observation of the reaction through a lens.

### DETAILED DESCRIPTION OF THE INVENTION

Several objects and advantages of our invention are to provide a cost-effective, accurate means of early (within 4 days of exposure) HIV-1 infection detection in whole blood that was based on the ability to immunochemically/ magnetically isolate and fluorescently label HIV-1 infected peripheral blood lymphocytes. Advantages of the invention, include, e.g.: 1. cell-bound antigen-based test closes the window period created by having to rely on the host immune system to produce antibodies against HIV-1 antigens to around four days; 2. Multi-purpose cartridge and fully automated incubator, magnetic separator and imaging system, permit operation by non-medically trained personnel; 3. Appearance of cell-bound gp120 parallels appearance of viral genetic material, enabling invention to detect HIV presence in same time period as “Gold Standard” PCR at a small fraction of the cost; 4. Functional design of the blood collection/ immunochemistry/ magnetic separation/ imaging cartridge permit complete, self contained, disposable unit that is much easier to handle than “gold standard” PCR test for viral genetic material; 5. Entire test procedure requires minutes to turn around compared with weeks for PCR; 6. Cost per test will be in tens of dollars rather than hundreds. Still further objects and advantages will become apparent from a consideration of the ensuing description and accompanying drawings.

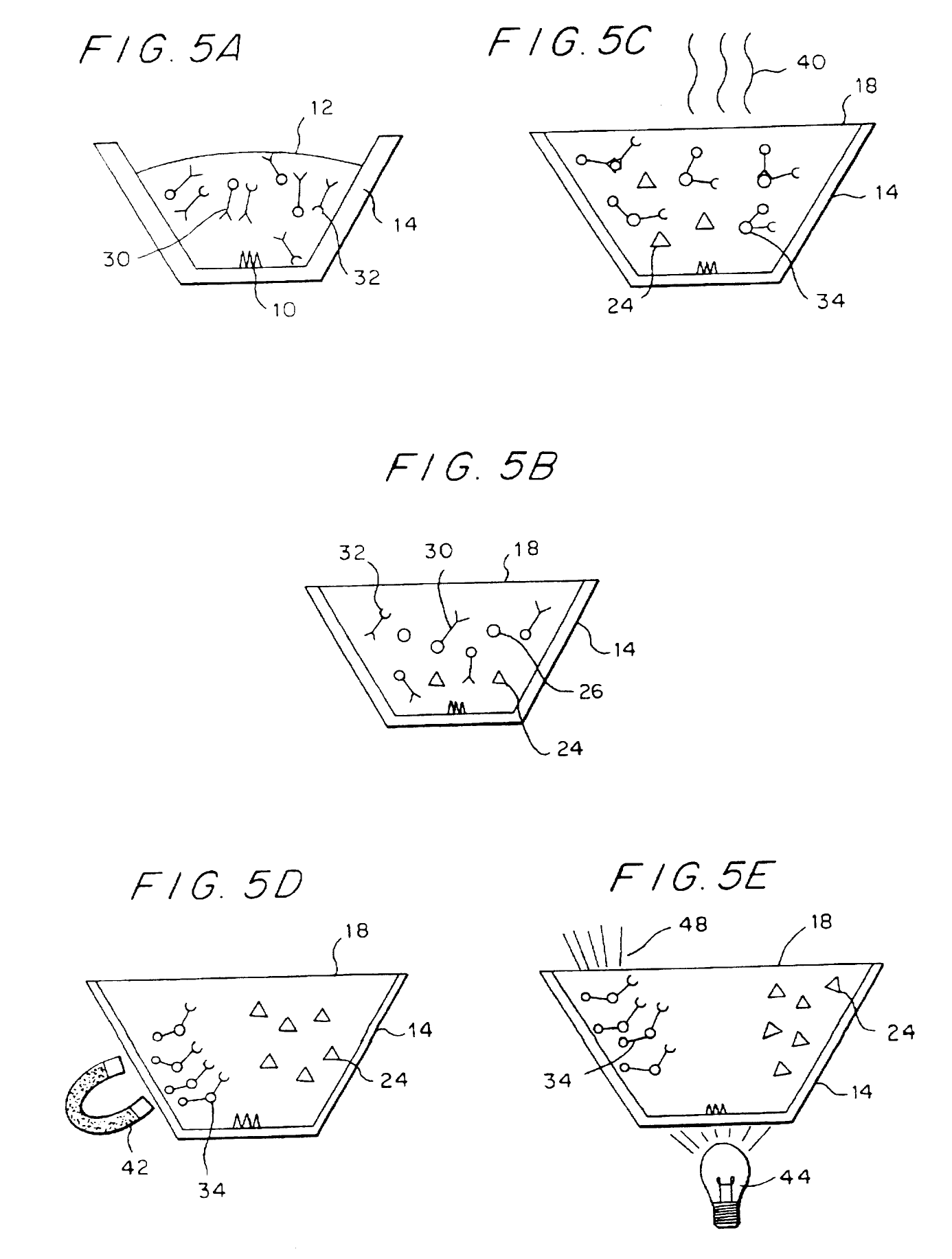

In one aspect of the present invention (see, e.g., FIG. 5), a sample of several drops of whole blood is diluted with murine anti-gp120 monoclonal antibodies coupled to paramagnetic microspheres approximately 0.5 cc Phosphate Buffered Saline (PBS). To the diluled sample is added the Murine anti-gp120 monoclonal antibodies coupled to paramagnetic microspheres, and the Fluorescein conjugated anti-gp120 polyclonal antibodies IgG. In the sample of diluted whole blood are a small number of HIV infected peripheral blood lymphocytes, bearing CD-4, also bearing numerous exposed gp120 antigens. The mixture of blood and antibodies after incubation for five minutes, both antibodies are non-competitively bound to each and every gp 120 antigen. This renders each HIV infected peripheral blood lymphocytes coated with both the murine anti-gp120 monoclonal antibodies coupled to paramagnetic micro spheres and the Fluorescein conjugated anti-gp120 polyclonal antibodies IgG. The uninfected peripheral blood lymphocytes remain uncoated by either of the antibodies. A vessel containing the mixture of incubated blood and reagents can be exposed to a strong magnetic gradient at a predetermined point on the outer surface of the reaction vessel. The magnetic field causes the migration of all HIV infected peripheral blood lymphocytes to the inner surface of the reaction vessel at the maximum point of concentration of the magnetic gradient, thus separating the HIV infected peripheral blood lymphocytes from the uninfected peripheral blood lymphocytes in the diluted whole blood sample. The magnetic separation takes approximately 20 seconds. After the designated time for magnetic separation to occur, the predetermined point of maximum magnetic concentration is illuminated by a suitable focused light source at 488 nm wavelength, causing all HIV infected peripheral blood lymphocytes, now aggregated at the at a predetermined point to glow at between 520-540 nm fluorescent light. Although there will be an excess of Fluorescein conjugated anti-gp120 polyclonal antibodies IgG unbound to HIV infected peripheral blood lymphocytes in the sample of diluted blood, the volume is sufficient and dilution of Fluorescein conjugated anti-gp120 polyclonal antibodies IgG adequate to provide only a low intensity diffuse background fluorescence as compared to the high intensity of cell bound Fluorescein conjugated anti-gp120 polyclonal antibodies IgG visible by fluorescence microscopy on the infected cells adhering to the inner surface of the reaction vessel wall. Likewise, the excess of magnetic particles unbound immunologically to cell surfaces will travel at a much greater velocity to the inner surface of the vessel wall, assuring that before any cell coated with magnetic particles arrive at the vessel wall, there will have formed a dark coating of unbound Murine anti-gp120 monoclonal antibodies coupled to paramagnetic micro spheres, against which the infected cells will adhere, also providing a nice contrast for the high density of glowing HIV infected peripheral blood lymphocytes.

Reagents can be obtained commercially, e.g., Immunodiagnostics, Inc., Murine Anti-gp120 HIV-1 mAb Coupled to Paramagnetic Microspheres. Monoclonal antibodies of mouse origin can be obtained commercially which are highly specific with high affinity to the gp120 HIV-1 glycoprotein. They are cross-reactive and cross neutralizing antibodies, which are covalently bonded to Paramagnetic Microspheres. Their coupling ratio is approximately 2.5 micrograms of protein per mg of magnetic microspheres. Specificity testing demonstrates that the Magnetic Murine anti-gp120 mAb binds recombinant gp120 (MN, IIIB) peroxidase conjugate as determined by ELISA. The biological activity is defined as the binding of these antibodies to CD-4 bearing, HIV-1 infected cells and HIV-1 infected human peripheral blood lymphocytes. Fluorescein Rabbit Anti-gp120 HIV-1 IIIB pAb IgG (e.g., Immunodiagnostics, Inc.) These Fluorescein conjugated anti-gp120 (HIV-1 IIIB) pAg IgG can be highly purified (95% pure) polyclonal IgG before use for FITC conjugation. The conjugate can then be further purified by gel exclusion chromatography. The specificity of this fluorescein conjugated pAb IgG can be defined by its binding to native and recombinant HIV-1 gp120 in Dot Blot assays and by its staining of cell surfaces in direct immunofluorescence assays. This reagent can be used for direct immunofluorescence assays. This reagent can be used for direct immuno fluorescent staining of cells in the 1:50 dilution range, while Dot blot assays with purified gp120 may be performed at a minimum dilution of 1:100.

Both monoclonal and polyclonal antibodies can be obtained (e.g., see above) which bind to the V3 loop of the HIV-1 envelope glycoprotein gp120 but which are not competitive, i.e., they attach to different regions of the V3 loop of gp120. This factor permits them to be used simultaneously for their specific and different purposes.

Further advantages of the above-described invention include, e.g., Cell—bound antigen-based test closes the window period created by having to rely on the host immune system to produce antibodies against HIV-1 antigens to around four days; Appearance of cell-bound gp 120 parallels appearance of viral genetic material, enabling invention to detect HIV presence in same time period as “gold standard” PCR at a small fraction of the cost; Entire test procedure requires minutes to turn around compared with weeks for PCR; Increased accuracy and low cost allow it to act as both screening and confirmatory test; This test can also be utilized in an automated format, utilizing a multi-purpose cartridge and fully automated incubator magnetic separator and imaging system, permitting operation by non-medically trained personnel; The test can be contained in a blood collection cartridge to permit complete, self contained, disposable unit that is much easier to handle than “gold standard” PCR test for viral genetic material.

Although the description above contains many specificities, these should not be construed as limiting the scope of the invention but as merely providing illustrations of some of the presently preferred embodiments of this invention. Various other embodiments and ramifications are possible within it's scope. For example, the method can be used to test for other viral infections by varying the antibodies combinations. In addition, other fluorochromes and detectable labels can be utilized. The method can be used test for water contamination, the method can be used to separate and identify cancer cells.

Another aspect of the present invention relates to an assay for determining the presence of a desired antibody or other binding partner in a test sample. In a preferred embodiment, the desired antibody is an antibody generated against an antigen coded for by a virus, e.g., HIV-1, HIV-2, HTLV, HTLV EBV, CMV, SIV, etc. In this preferred embodiment, the antibody is a neutralizing antibody or an antibody able to interfere with infection of a cell by the virus. In this aspect of the invention, the presence of the antibody in the test sample is determined by its ability to interfere with infection of a target cell by a virus.

To perform this aspect of the invention, a target cell is contacted with a virus capable of infecting the cell. The step of contacting can be achieved, e.g., by combining the target cell and virus in a receptacle, such as a test tube, a slide well, a tissue culture flask, etc. Typically, the step of contacting is performed in a liquid phase; however, a solid phase can also be used.

A target cell which is useful in the present invention is one which is susceptible or receptive to being infected by a desired virus, e.g., HIV-1. Various cells can be used, including, primary cells such as CD4(+)T cells or splenic cells, T cell lines, lymphoblastoid cell lines, H9, C8166, Molt, Molt-4, CEM, Jurkat, preferably, CEMX174. See, e.g., Virology, 236:208-212, 1997. Also, MAGI-CCR5

A target cell can be contacted with the virus under conditions effective to achieve infection of the cell with the virus. By this phrase, it is meant any factors (e.g., cofactors, protein, cytokines, ions), environments, ingredients, etc., useful for the virus to infect the cell, e.g., to attach to the cell, enter the cell, and pirate the cell's machinery for its own benefit. These conditions can be routinely determined, e.g., including optimizing pH, temperature, salts, buffers, virus concentrations, cell numbers, etc. The effective conditions also can mean a media or other liquid environment in which invasion of the cell by a virus is accomplished. A media can include growth factors and other compounds which facilitate the virus's entry into a cell. Any suitable buffer system can be used, including, e.g., PBS, Tris, sodium citrate, etc., borate, etc.

The combination of the target cell and virus can be referred to as a mixture. This means, e.g., that the target cell and virus are present together, e.g., in the same receptacle, in such a manner that infection can occur. Thus, when the target cell is contacted with the virus, a mixture is formed. As mentioned the contacting is accomplished in a suitable environment for infection and expression of an antigen associated with viral infection, e.g., gp120, gp41, etc.

A feature of the invention is detection of the viral antigen on the cell surface and detection of agents which interfere with its expression. Detection of the antigen can be achieved directly or indirectly. In one embodiment, one or more receptors for a viral antigen, e.g., CD4, CKR5, CC-CKR5, CCR5, CKR2, CKR2B, CKR3, CKR4, CXCR4, CCR2, fusin, etc (See, e.g., J. Virol., 71:1657-1661, 1997; Dean et al., Science, 273:1856-1862, 1996) can be coupled to a surface (e.g., a magnetic particle, bead, or miscrosphere) and then used to capture cells expressing the viral antigen. In another embodiment, an indirect means of capture is used. For example, a first binding partner, specific for an antigen coded for by the virus which is expressed on the cell upon viral infection, is added to the mixture. The term “specific” has its normal art-recognized meaning, e.g., it is has a higher affinity for the viral antigen than other antigens present in the mixture. The binding partner can be added at the same time as the virus, or, after the virus has been added to the mixture. The conditions used are those which permit (e.g., “effective”) for the binding partner to attach to the viral antigen as it is expressed on the cell surface, or otherwise displayed by the cell.

The binding partner can be any agent which recognizes the viral antigen, e.g., aptamers, PNA structures, peptides, small molecules, antibodies (monoclonal, polyclonal, chimeric, single-chain, divalent, disulfide-stabilized Fv fragments, etc.), receptors for a viral antigen (e.g., CKR5, CD4), etc. Methods for making antibodies are well-known in the art Antibodies can also be obtained from commercial sources, e.g., Immunodiagnostics, Waltham, Mass.

Once the binding partner is attached to the viral antigen on the cell surface, the cell can be captured directly if the binding partner is attached to a substrate, e.g., a magnetic particle, or, it can be captured by using a second binding partner which is able to specifically recognize the first binding partner, i.e., specifically bind to among a mixture of molecules, antigens, agents, antibodies, etc. As mentioned, above “specific for” has its art-recognized meaning. The second binding partner is preferably attached to a substrate, e.g., a latex bead, a glass slide, a microwell, a magnetic bead or particle, etc. Attachment of the binding partner to the substrate can be accomplished according to conventional methods. See, e.g., U.S. Pat. No. 5,543,289; Luk and Lindberg, J. Immunol. Methods, 137:1-8, 1991.

Another aspect of the present invention relates to the use of a viral receptor as the primary binding partner utilized to capture the target material, e.g., a cell infected with a virus. For instance, in the case of HIV, CD4, a receptor for HIV, is a preferred reagent for several reasons. It is a universal primary binding site for subtypes, strains, and clades of the HIV virus and is also on most HIV receptive cells. Because an already HIV-infected cell expresses the gp120 envelope protein diffusely distributed over its entire membrane surface, and because purified (recombinant or from natural sources) CD4 has a high specificity and affinity for gp120, it useful to capture material containing gp120. For purification, see, e.g., U.S. Pat. No. 5,603,933; Deen et al., Nature, 331:82-84, 84-86, 1988; Watanabe et al., Nature, 335:267-270, 1989. In such an embodiment, CD4, for instance, can be used indirectly to capture target material, in accordance with the methods described above and below. For instance, CD4 can be conjugated to a moiety, e.g., FITC, and employed to capture, e.g., HIV-infected cells in mixture which contains both infected and uninfected cells. Magnetic particles containing anti-FITC antibody can be used in turn to label the cells coated with the CD4-FITC. the mixture can then be passed through a separator column causing positive selection of all the HIV-infected cells in the mixture, while depleting the uninfected cells. After depleting uninfected cells from the mixture, a separation column can be removed from the magnetic filed and the HIV-infected cells eluted with PBS or another suitable buffer. The FITC-conjugated CD4 labeled cells can then be fixed and counted by standard flow cytometry. Cells infected with other viruses can be selected analogously.

In a preferred embodiment, the second binding partner is attached to a magnetic particle (bead, microsphere, etc.), e.g., as described in U.S. Pat. No. 5,411,863; U.S. Pat. No. 5,543,289. A magnetic particle can be comprised of any effective type, e.g., ferromagnetic, supermagnetic, paramagnetic, and superparamagnetic. A preferred particle is comprised of iron oxide and polysaccharide. A preferred magnetic bead has a diameter which is less than the diameter of the cell which is to be captured, e.g., about 1-300 nm, about 5-200 nm, about 10-150 nm, preferably, about 20-150 nm, more preferably, about 50-120 nm. Preferably, the magnetic beads are of a sufficient size that they can form a coating around the cell, e.g., having more than one bead attached to the cell, such as about 10 beads, about 100 beads, about 1000, or about 100-1000 etc. These beads be manufactured or commercially obtained e.g., Miltenyi Biotech, Germany.

The second binding partner is selected for its ability to specifically bind to the first binding partner, i.e., recognize and attach to it with a higher affinity than other components in the cell mixture. The second binding partner can be of any material, e.g., those described for the first binding partner. In an embodiment, the first binding partner comprises a moiety which is recognized specifically the second binding particle. The moiety can be attached conventionally to the binding partner. Such a moiety can be, for instance, a hapten or detectable label, such as a fluorochrome, e.g., FITC, TRITC, R-phycoerythrin, Quantum Red, or Cy3, gold, ferritin, biotin, avidin, streptavidin, green fluorescent protein GFP (Chalfie et al., 1994, Science, 263:802; Cheng et al., 1996, Nature Biotechnology, 14:606; Levy et al., 1996, Nature Biotechnology, 14:610), alkaline phosphatase, peroxidase, HRP, urease, an arbitrary hapten, etc.

In one embodiment, a first binding partner can be an anti-gp120 antibody conjugated to FITC and the second binding partner can be an anti-FITC antibody. In another embodiment, the first binding partner can be a receptor for a viral antigen expressed on the cell surface upon viral infection (e.g., CD4, CKR5, fusin, etc). A second binding partner can be selected which is specific for the viral receptor. Such binding partner can be an antibody which recognizes an epitope, etc., on the receptor. The receptor can also comprise a moiety, as mentioned above, and the second binding partner can be an agent which recognizes the moiety, e.g., where the first binding partner is a receptor conjugated to FITC, the second binding partner can be an anti-FITC antibody preferably coupled to one or more paramagnetic microspheres.

A second binding partner can be added at the same time as when the virus is contacted with the cell, or it can be added later, e.g., after cell contact, after addition of the first binding partner. Preferably, a virus or mixture of viruses are added to the cells and then incubated for a sufficient amount of time for the virus to infect the cell and for the cell to display evidence of such infection (e.g., surface expression of gp120 or gp41). the first and second binding partner can then be added in subsequent and sequential steps. After each addition, optionally, an incubation period is utilized providing adequate time for the binding partner to attach to its substrate. Such times can be routinely determined. As a result of the above-mentioned steps, a cell-antigen-first binding partner-second binding partner combination is formed. The antigen-first binding partner-second binding partner combination can be referred to as a complex when at least these three components are joined together and attached to a cell. Preferably, the complex included a magnetic particle, e.g., when the second binding particle is attached to it. When a magnetic particle is included in the complex, separation can be achieved conventionally by a magnetic field. See, e.g., U.S. Pat. Nos. 5,541,072; 5,543,289; 5,238,810; 5,196,827; 4,731,337, e.g., by positive selection. For instance, in one embodiment, a chamber having an inlet and outlet is filled via the inlet with a sample. The sample contains, e.g., the cells (such as HIV-infected cells) coated with paramagnetic microspheres. A material which is capable of expressing a magnetic field surrounds the filled chamber. A magnetic field is applied to the column, retaining the cells coated with the paramagnetic beads, and allowing the uncoated cells to flow out through the outlet of the chamber. The infected, coated cells can be eluted by releasing the magnetic field. The chamber can comprise any material or matrix, including materials or matriices capable of expressing a magnetic field. Such technology is conventional. U.S. Pat. No. 5,411,863 describes an apparatus, system, and particles which can be used in the present invention.

A related aspect of the present invention, is the identification of agents which interfere, modulate, prevent, or enhance, viral infection of a cell. Such agents can be antibodies, small molecules, aptamers, ribozymes (hammerhead, intron, hairpin, etc.), proteins (cytokines, growth factors, cytokinin antagonists, etc), antiviral agents (proteases, nucleotides, etc.), chemokines, chemokine antagonists (e.g., antagonists, including antibodies to, e.g., RANTES, MIP-1a, MIP-1b). To accomplish this facet of the invention, the suspected agent can be added to the mixture as described above and the number of cells captured in the presence or absence of the agent tested or measured. The cells can be pretreated with the agent, e.g., to identify agents which interfere with viral infection after the virus has entered the cell. The agent can be added to the mixture at the same time as the virus, e.g., to identify agents which interfere with viral attachment to the cell or which disable the virus before attachment.

Various samples can be used in the present invention, including, any material suspected of containing cells or agents which interfere with viral infection or virus, itself, such as blood, lymph, tissues, organs, in vitro cell culture, urine, saliva, sweat, water samples (e.g., for testing drinking water quality), cell culture media, FBS, serum, feces, food, saline solutions, etc. Such material can be derived from any source or species, including invertebrates, vertebrates, bacteria, mammals, such as humans, apes, monkeys, etc., mollusks, insecta, etc.

Sensitivity can be increased in the above- and below-mentioned assays and tests by detecting viral antigen before it is expressed on the cells surface, e.g., as it is synthesized and/or processed within an infected cell. By detecting viral antigen within the cell, virus infection can be detected much earlier, e.g., about 4 hrs, about 4-8 hrs, about 8-12 hr, about 12-15 hrs, or about 15-18 hrs, or about 18-24 hrs, after infection of the cell with virus. Early detection is especially advantageous when performing screens for agents (antibodies, antagonists, etc.) which interfere with infection; by decreasing detection time, the number of samples that can be screened in a given amount of time is increased. High throughput screening can thus be achieved in accordance with the present invention by detecting the presence of internals antigen, e.g., in the endoplasmic reticulum, golgi, vesicles, etc., including antigens expressed on the cells surface, such as gp120, but also other antigens whose ultimate location is intracellular. Intracellular detection can be achieved in a variety of ways, including, e.g., fixation of cells (using conventional agents and methods, such as formaldehyde, paraformaldehyde, glutaraldehyde, alcohols, etc) followed by permabilization (using conventional; agents and methods, such as triton, triton X-100, tween-20, etc. As is typical, the fixative and permeabilizing agent can be removed after the cells are fixed and permeabilized. Thus, the present invention relates to a method of detecting and/or separating cell infected with a virus, comprising various steps including, contacting a target cell with a virus capable of infecting the cell, under conditions effective for achieving infection of the cell with the virus, a preferred virus is HIV, SIV, or related viruses; fixing and permeabilizing the virus-infected cells; adding to the fixed and permeabilized cells, a first binding partner specific for an antigen coded for by the virus, which viral antigen is expressed on the surface of the cell upon viral infection, under conditions effective for the first binding partner to bind to said viral antigen on the inside of said fixed and permeabilized cell, a preferred first binding partner a polyclonal antibody, or cocktail of monoclonal antibodies that bind to gp120, gp40, etc., which antibodies are conjugated to a tag, such FITC; adding to the result of the latter, a second binding partner specific for the first binding partner and attached to a magnetic bead or a microbead, under conditions effective for the second binding partner to bind to the first binding partner, when the first binding partner is bound to the antigen expressed by the cell, to form a complex, where the second binding partner is preferably anti-FITC or reactive with the tag portion of the first binding partner, and; separating target cells containing the complex, whereby said separation is achieved by a magnetic field.

This method can also be used in conjunction with methods of identifying agents which interfere with viral infection of a cells. Thus, the invention relates to a method of identifying an agent which interferes with viral infection of a cell, including steps the following steps, which can be performed in any desired order, sequence, or combination. Steps can also be added or deleted. For example: Contacting a test cell with a virus capable of infecting the test cell, under conditions effective for achieving infection of the cell with the virus, to form a mixture. Adding a test agent suspected of interfering with viral infection (e.g., an antibody against the virus coat, the cell receptor, or ligands which interfere with binding of the virus to the cell surface receptor) for an amount of time effective for the agent to interfere with virus infection. The agent can be added simultaneously, before, or after virus treatment. Fixing and permeabilizing the cells, according to methods which are conventional in the art (see above and standard textbooks). Adding to the fixed and permeabilized cells, a first binding partner specific for an antigen coded for by the virus, which antigen is expressed on the surface of the test cell upon viral infection, under conditions effective for the binding partner to bind to the viral antigen on the cell surface when said antigen is still inside the cell, e.g., in the ER, where the virus is preferably HIV, SIV, or a derivative thereof, and the first binding partner is preferably a cocktail of monoclonal antibodies or a polyclonal antibody against gp120, gp40, etc. and the antibody is attached to a tag, such as FITC. Adding to the resultant mixture formed in the latter steps, a second binding partner specific for the first binding partner and to a magnetic bead, under conditions effective for the second binding partner to bind to the first binding partner, when the latter is bound to the viral antigen expressed by the test cell, to form a complex, where the antibody is preferably anti-FITC, or another antibody directed to the tagged portion of the first binding partner; separating test cells containing said complex, whereby said separation is achieved by a magnetic field. Performing the infection in the absence or presence of the test agent and determining whether the cell infectivity is affect, e.g., reduced, decreased, delayed, by the agent. A related aspect of the present invention involves isolation of viruses from samples, e.g., HIV from plasma. For example, HIV can be isolated from plasma by coupling 2 nm magnetic microbeads with anti-gp41/ and/or anti-gp 120. The technique will enrich the virus concentration by immunomagnetic separation with no loss of virus through centrifugation and permit efficient separation from plasma inhibitors of PCR. The same methods described above for cells can therefor can be used for viral isolation. However, magnetic microbeads, e.g., from about 0.5-10 nm, preferably 1-5 nm can be used.

Flow cytometry can be accomplished conventionally. For example, in one embodiment, the coated cells are eluted from the magnetic separation apparatus. See, e.g., U.S. Pat. No. 5,411,863. Such cells can then be subjected to flow cytometry according to any method. See, e.g., Hiebert, R. D., “Electronics and Signal Processing”, Flow Cytometry and Sorting, Second Ed., Wiley-Liss Inc., pp. 127-155, 1990; M. Loken et al., “Two-Color Immuunofluorescence using a Fluorescence-Activated Cell Sorter”, The Journal of Histochemistry and Cytochemistry, 25(7):899-907, 1977; Sutherland et al., “Sensitive detection and enumeration of CD34 cells in peripheral and cord blood by flow cytometry”, Exp. Hematol., Vol. 22, pp. 1003-1010, 1994; V. Cacheux et al., “Detection of 47XYY Trophoblast Fetal Cells in Maternal Blood by Fluorescence in situ Hybridization after Using Immunomagnetic Lymphocyte Depletion and Flow Cytometry Sorting”, Fetal Diagn. Ther., Vol. 7, pp. 190-194, 1992; P. N. Dean, “Commercial Instruction”, Flow Cytometry and Sorting, Second Ed., Wiley-Liss, Inc., pp. 171-186, 1990; T. Lindmo et al., “Flow Sorters for Biological Cells” Flow Cytometry and Sorting, Second Ed., Wiley-Liss, Inc., pp. 145-169, 1990; H. B. Steen, “Characteristics of Flow Cytometers” Flow Cytometry and Sorting, Wiley-Liss, Inc., pp. 11-25, 1990; M. R. Melamed et al., “An Historical Review of the Development of Flow Cytometers and Sorters”, Flow Cytometry and Sorting, Second Ed., Wiley-Liss, Inc. 1990, pp. 1-9, 1990; Gottlinger et al., “Operation of a Flow Cytometer”, Flow Cytometry and Cell Sorting, A. Radbruch Ed., pp. 7-23, 1992; Schols et al., “Flow Cytometric Method to Demonstrate Whether Anti-HIV-1 Agents Inhibit Virion Binding to T4 Cells”, Vol. 2, pp. 10-15, 1989; Sallusto et al., Science, 277:2005-2007, 1997; U.S. Pat. Nos. 5,602,349; 5,675,517; 5,665,557; 5,641,457; and 5,582,982.

As mentioned, these methods can be applied to the separation of cancer cells, microorganisms, cells expressing antigens of interest, from mixed populations. For example, a method of separating a microorganism having a cell-surface antigen comprises combining (a) a first antibody recognizing said cell-surface and attached to a magnetic particle; (b) a second antibody recognizing said antigen and attached to a detectable label; and (c) a sample containing said microorganism, to form a mixture; incubating said mixture under conditions effective for binding of said antibodies to said antigen to form a complex, said complex comprising said first and second antibody bound to said microorganism, and moving said magnetic particle to a predetermined point on a reaction vessel holding said mixture, whereby said moving is accomplished by a magnetic field acting on said magnetic particle resulting in separating said microorganism, and said moving is accomplished without removing unbound first antibody and second antibody from said mixture.

Another example further comprises detecting the label of said second antibody bound to said microorganism.

A further method of separating cancer cells in a mixture of cancer and normal cells comprises combining (a) a first antibody recognizing a cancer antigen on a surface of said cancer cell and attached to a magnetic particle; (b) a second antibody recognizing said cancer antigen on a surface of said cell and attached to a detectable label; and (c) a sample containing said cancer cells; incubating said mixture under conditions effective for binding of said antibodies to said cancer antigen to form a complex, said complex comprising said first and second antibody bound to said cancer cell on said magnetic particle, and moving said mixture under conditions effective for binding of said antibodies to said cancer antigen to form a complex, said complex comprising said first and second antibody bound to said cancer cell, and moving said magnetic particle to a predetermined point on a reaction vessel holding said mixture, whereby said moving is accomplished by a magnetic field acting on said magnetic particle resulting in separating said cancer cell from said mammal cells, and said moving is accomplished without removing unbound first antibody and second antibody from said mixture.

An additional method further comprises detecting the label of said second antibody bound to said cancer cells.

A method of separating cancer cells expressing a cell-surface cancer antigen from normal cells, comprises: a) combining an effective amount of an anti-cell-surface cancer antigen antibody attached to a detectable label, an effective amount of an antibody specific-for said detectable label, and an aqueous sample containing cancer cells displaying said cell-surface cancer antigen to form a mixture, wherein said antibody specific-for said detectable label is attached to a magnetic particles; b) incubating said mixture under conditions effective for binding of said anti-cell surface cancer antigen antibody to said cell-surface cancer antigen, and, for binding of said antibody specific-for said detectable label to said detectable label attached to said anti-cell surface cancer antigen antibody to form a complex, wherein said anti-cancer antibody is bound to said cell-surface antigen displayed on a cancer cell; and c) separating said complex comprising said cells expressing said cell surface antigen and magnetic particles by applying a magnetic field to said mixture, whereby said complex is retained by said magnetic field.

A further method of separating cells expressing a cell-surface cancer antigen from normal cells, comprises a) combining an effective amount of an anti-cell-surface cancer antigen antibody attached to a magnetic particle and an aqueous sample containing cancer cells displaying said cell-surface viral antigen and normal cells, to form a mixture; b) incubating said mixture under conditions effective for binding of said anti-cell surface cancer antigen antibody to said cell-surface cancer antigen displayed on said cancer-infected cells to form a complex; and c) separating said complex comprising said cells expressing said cell surface antigen and magnetic particles by applying a magnetic field to said mixture, whereby said complex is retained by said magnetic field.

For other aspects of the polypeptides, antibodies, etc., reference is made to standard textbooks of molecular biology, protein science, and immunology. See, e.g., Davis et al. (1986), Basic Methods in Molecular Biology, Elsevir Sciences Publishing, Inc., New York; Molecular Cloning, Sambrook et al.; Current Protocols in Molecular Biology, Edited by F. M. Ausubel et al., John Wiley & Sons, Inc; Current Protocols in Human Genetics, Edited by Nicholas C. Dracopoli et al., John Wiley & Sons, Inc.; Current Protocols in Protein Science; Edited by John E. Coligan et al., John Wiley & Sons, Inc.; Current Protocols in Immunology; Edited by John E. Coligan et al., John Wiley & Sons, Inc.

### DETAILED DESCRIPTION OF TEST KIT

FIG. 1 shows the Cartridge Antigen Test (CAT), comprising a cartridge 16 and a clear rectangular piece of plexiglas, ⅜″thick, 2 wide, and 3″long. The well 14, a ¼″ deep central hemispherical depression in the middle of the cartridge 16, holds the micro-baggy containing the mixture of reagents 12 and three micro-lances 10. The well 14 is covered by a clear mylar strip 18 and adhesive fastener 20. A bar code strip 22 is near the bottom of the cartridge 16.

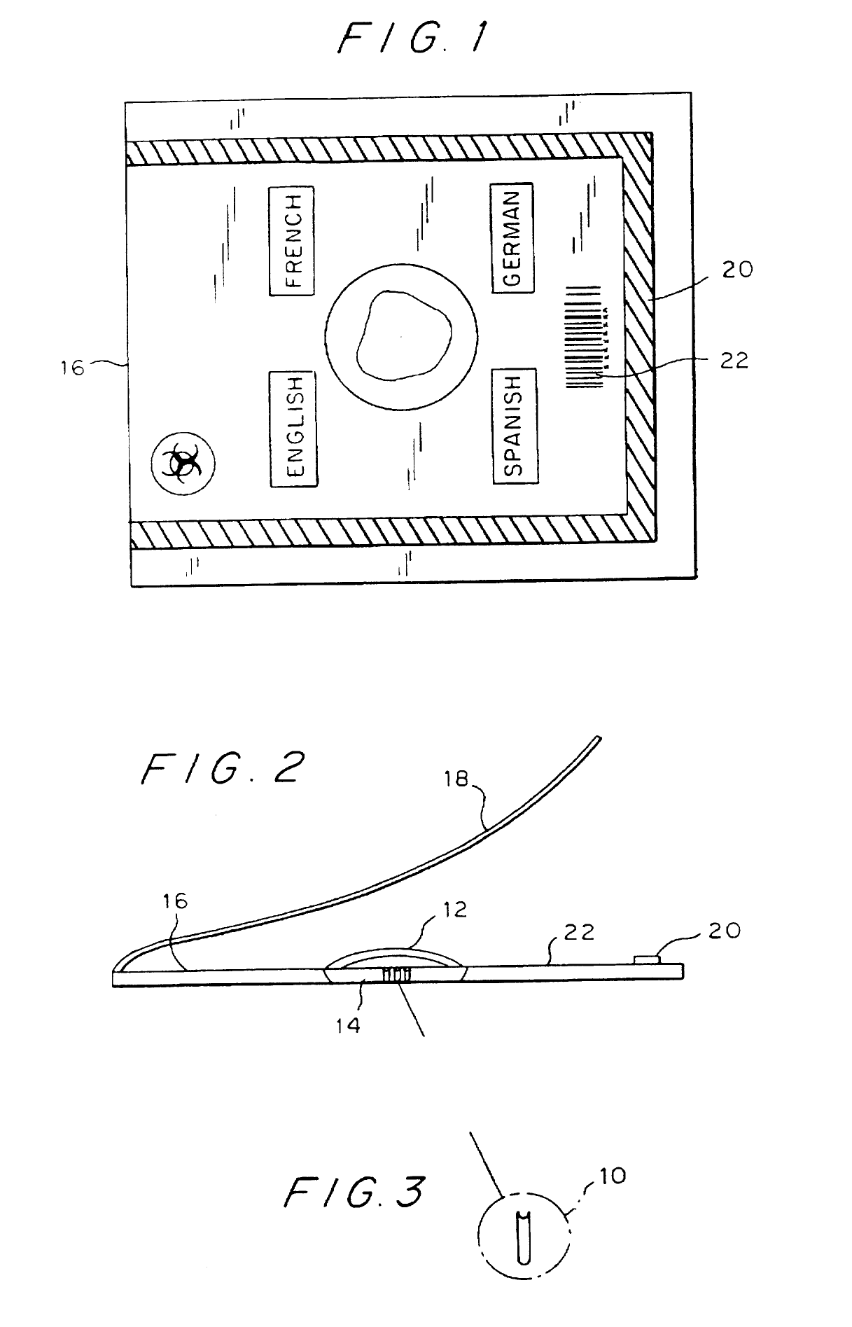

FIG. 2 shows that the well 14 is clear and transparent on the sides, top and bottom, allowing light to pass through the reagent/blood mixture.

FIG. 3 shows one of the three micro-lances 10 which protrude from the bottom of the center of the depression or well 14. Sitting just above the three micro-lances 10 is a micro-baggy containing the mixture of reagents 12.

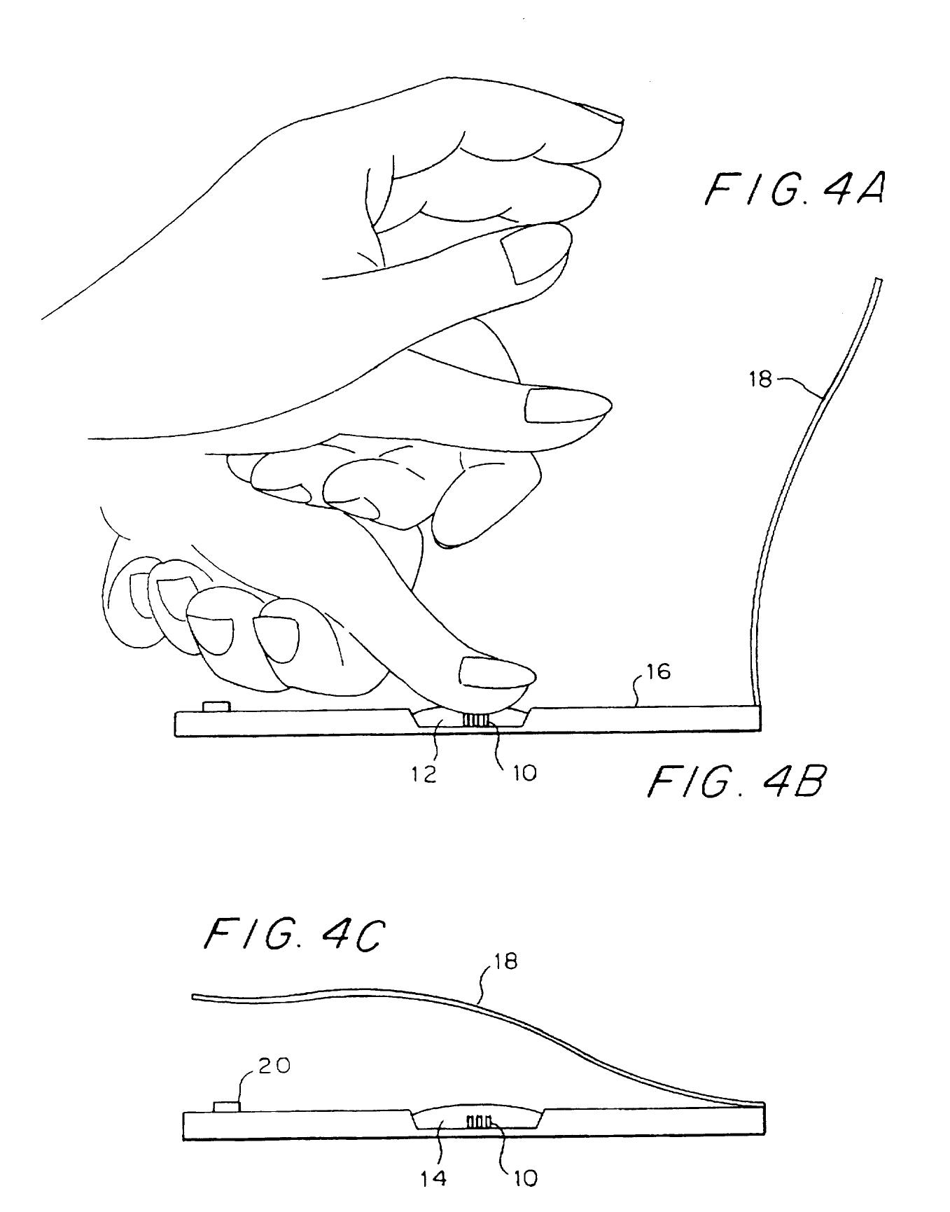

FIG. 4A shows how a test subject holds his/her hand above the well 14 of the cartridge 16.

FIG. 4B illustrates how the pressing of the thumb on the micro-baggy containing the mixture of reagents 12 above the three micro-lances 10 will cause the test subject to bleed, the blood to be mixed with the reagents. FIG. 4C shows how the cartridge 16 is sealed after collection with the clear mylar strip 18 by lowering the mylar strip 18 into contact with the adhesive strip 20.

FIG. 5A is a side view of the well 14 before the test subject bursts the micro-baggy containing the mixture of reagents 12. The well 14 contains two reagents needed for the magnetic separation and fluorescent identification: antibodies coupled to paramagnetic microspheres 30 and antibodies coupled with a fluorochrome 32.

FIG 5B is a side view of the well 14 covered with the clear mylar strip 18, with the wholl blood sample and reagents prior to incubation.

FIG. 5C is a side view of the well 14 covered with the clear mylar strip 18, after mixing the whole blood sample with the reagents. Incubation 40 is applied to the cartridge 16 and the uninfected peripheral blood lymphocytes 24 remain unaffected by the reagents. The incubation 40 produces antibodies noncompetively bound to infected peripheral blood lymphocytes 34.

FIG. 5D shows the well 14, being exposed to a strong magnetic gradient 42. The magnetic field caused the migration to the inner surface of the well 14 of all the antibodies noncompetively bound to infected peripheral blood lymphocytes 34 to the point of concentration of the magnetic gradient 42, thus separating the antibodies noncompetively bound to infected peripheral blood lymphocytes 34 from the uninfected peripheral blood lymphocytes 24. The magnetic separation takes approximately 20 seconds.

FIG. 5E shows a side view of the well 14 after the magnetic separation has occurred. The predetermined point of maximum magnetic concentration is illuminated by a suitable focused light source 44, for example, at 488 nm wavelength, for FITC, causing all antibodies noncompetively bound to infected peripheral blood lymphocytes 34 now aggregated at the predetermined point to glow 48 at between 520-540 nm fluorescent light. The reaction can then be viewed through a microscope or lens of an imaging system.

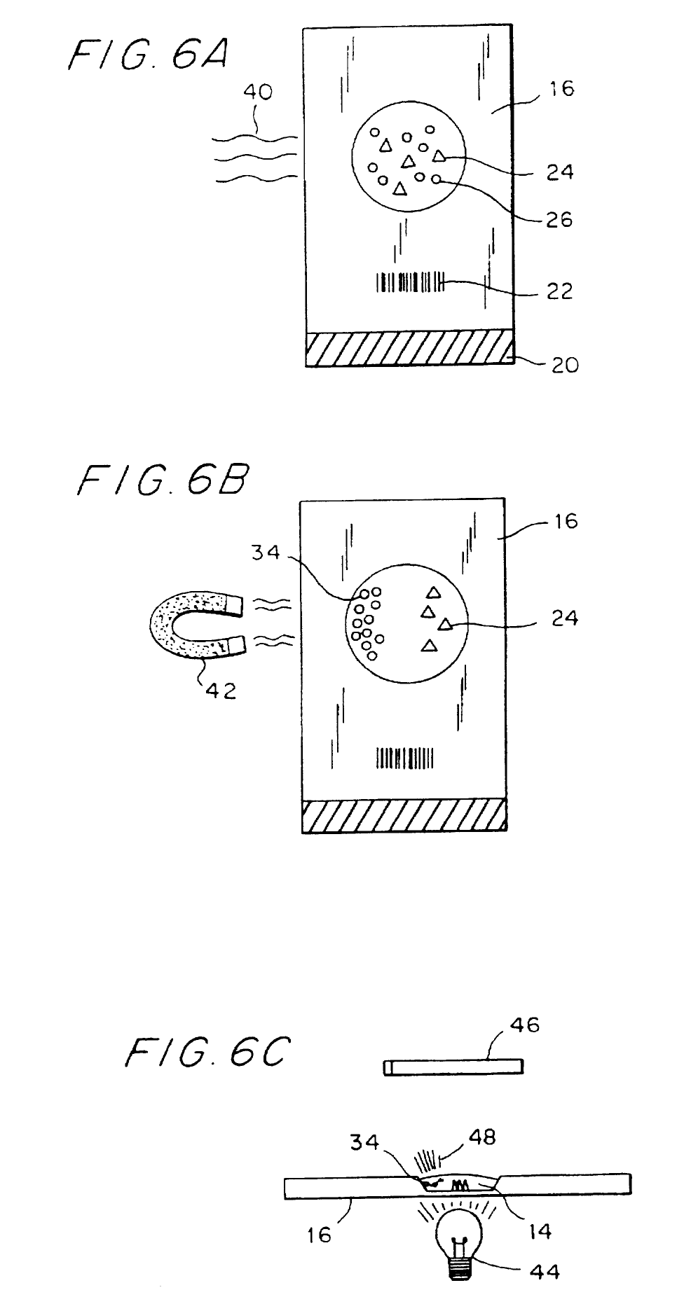

FIG. 6A shows a top surface view of the cartridge 16. FIG. 6B shows the antibodies noncompetively bound to infected blood lymphocytes 34 being separated from the uninfected peripheral blood lymphocytes 24 by the magnetic field to the concentration pont of the magnetic gradient 24.

FIG. 6C is a side view of the cartridge 16 and shows how the focused light source 44 is directed through the bottom of the well 14 and the lens 46 placed above the well 14 to view the glow 48 from the reaction.

To use the invention, a test subject presses his/her thumb or finger down onto the micro-baggy containing the mixture of reagents 12 on the CAT. The micro-baggy containing the mixture of reagents 12 bursts. The three micro-lances 10 puncture the thumb or finger causing the individual to bleed. The reagents in the bubble and the blood mix. The clear mylar strip 18 is pulled down and fastened by adhesive fastener 20, sealing the well 14 containing the blood and the reagents.

In the specific embodiment, two reagents must be present in the well to complete both the magnetic separation of the targeted micro-organism and the flourescent identification of their presence: the first reagent must comprise anti-bodies coupled to paramagnetic microspheres 30 and the second must consist of anti-bodies coupled with a Fluorochrome 32. Both reagents will bind themselves to the infected or target antigen-coated cells during the incubation 40.

The mixture in the sealed cartridge 16 is incubated for 3 to 5 minutes at 37 degrees Centigrade. The cartridge 16 is then moved to a viewing platform. A strong magnetic gradient 42 is applied to the side of the well 14. The magnetic field causes the target antibodies, noncompetively bound to infected peripheral blood lymphocytes 34, to separate from the other untargeted cells to a fixed point where the magnetic gradient 42 is concentrated. A forced light source 44, measuring 488 nm is passed through well 14 and the blood and reagent mixture. The focused light source 44 causes antibodies noncompetively bound to infected peripheral blood lymphocytes 34 to glow 48 at the specific emission frequency determined by the specific fluorochrome. The reaction can be viewed through a lens 46 or predetermined coordinates of the magnetic gradient 42 with the highest concentration at the inner surface of the well 14 where the antibodies noncompetively bound to infected peripheral blood lymphocytes 34 will be located. If there is no glow then the result is negative, and if there is a glow 48 the result is positive.

The test subject is identified by the bar code strip 22 attached to the cartridge 16.

Accordingly, it can be seen that the invention simplifies the procedures of blood collection, reagent mixing, patient tracking and test reading by unifying all steps into one functional unit. The positioning of the micro-baggy containing the mixture of reagents 12 above the three micro-lances 10 allows for blood collection and mixing with the reagents in one step. The clear mylar strip 18 is used to cover the exposed well 14 and the cartridge 16 is incubated 40 at 37 degrees Centigrade.

The invention works with two reagents. The first reagent consists of antibodies coupled to paramagnetic microspheres 30 so that the infected peripheral blood lymphocytes 26 can be separated from uninfected peripheral blood lymphocytes 24 by applying a magnetic gradient 42. The magnetic field generated by the magnetic gradient 42 will cause the antibodies coupled to paramagnetic microspheres 30 attached to the infected peripheral blood lymphocytes 26 to be drawn to a predetermined location of the interior wall of the well 14.

The second reagent consists of antibodies coupled with a fluorochrome 32 so that the infected peripheral blood lymphocytes 26 can be identified if present by applying a focused light source 44 on the well 14 causing the infected peripheral blood lymphocytes 26 to glow at the specific emission frequency determined by the specific fluorochrome. The well 14, covered with a clear mylar strip 18, allows the cartridge 16 to move around and allows the test reaction to be viewed through a lens 46.

List of Reference Numerals

10 Three micro-lances

12 Micro-baggy containing the mixture of reagents

14 Well

16 Cartridge

18 Clear mylar strip

20 Adhesive fastener

22 Bar code strip

24 Uninfected peripheral blood lymphocytes

26 Infected peripheral blood lymphocytes

30 Antibodies coupled to paramagnetic microspheres

32 Antibodies coupled with a Fluorochrome

34 Antibodies noncompetively bound to infected peripheral blood lymphocytes

40 Incubation

42 Magnetic gradient

44 Focused light source

46 Lens

48 Glow

Although the description above contains many specificities, these should not be construed as limiting the scope of the invention, but as merely providing illustrations of some of the presently preferred embodiments of this invention. Various other embodiments and ramifications are possible within its scope. For example, a variety of immunochemical reactions used in diagnosing infectious diseases can be done using a cartridge 16, by substituting the reagents in the micro-baggy containing the mixture of reagents 12. An automated cartridge processor can use the CAT to perform test outside of the environment of a high tech laboratory and can be operated by an untrained personnel. Tests that do not require magnetic separation can be performed using this invention.

Thus, the scope of this invention is determined only by the appended claims and their legal equivalents, rather than by the examples given.

### EXAMPLES

HIV-1 Isolation System

For the numerous instances when it is desirable to separate HIV-1 infected cells from a mixture of uninfected cells, an HIV-1-infected cell isolation system utilizing immunomagnetic separation can be used. The mixture of infected and uninfected cells is washed, centrifuged and resuspended in PBS. A polyclonal anti-GP 120-FITC is introduced into the resuspended cells and incubated for 10 minutes. The cells are separated by centrifugation and washed. Anti-FITC microbeads are used to separate the fluorescently labeled HIV-1-infected cells using positive selection columns. Flow cytometry is used to quantitate the separated HIV-1-infected cells.

This same isolation system can be used with other virally-infected cells, such as SIV or HTLV.

HIV-1 Neutralization Assay

Utilizing the principle of immunomagnetic separation of HIV-infected cells, a neutralization assay can be used to determine the quantity of neutralizing antibody activity in sera. A positive control is established by inoculating receptive CEMX 174 cells, or another viral receptive cell line, such as MAGI CCR5) in suspension with an isolate of HIV-1 (89-6). After two hours, the inoculating virus can be separated from the cells by centrifugation, washing, centrifugation, and resuspension in PBS. After two days of incubation, the cells. After two days of incubation, the cells are centrifuged, washed, and resuspended in PBS. After 2 days of incubation, the cells are separated by centrifugation, washed and resuspended in PBS. Anti-GP120-FITC is introduced into the resuspended cells and incubated for 10 minutes. The cells are lifted, separated by centrifugation and washed. Anti-FITC Microbeads are used to separate the fluorescently labeled HIV-1 infected cells using positive selection columns. The HIV-infected cells are then quantified using standard flow cytometry or manually with hemacytometer..

The procedure for determining neutralizing activity of sera is performed by adding serially diluted sera specimens to the mixture of virus and CEMX174 cells and incubating for the same time as used for the positive control. Anti-GP 120-FITC and Anti-FITC Microbeads are used in the same way as in the positive control to separate and enumerate the HIV-1-infected cells. The neutralizing activity of each serum specimen is determined by the difference from the positive control in the quantity of HIV-infected cells isolated after treatment and incubation of cells and virus with neutralizing sera.

This same assay can be performed with other virally-infected cells, such as SIV or HTLV-1. The assay can also be performed to test engineered neutralizing monoclonal for their ability to interfere with viral reproduction.

HIV-1 Drug Screening Assay

Utilizing the principle of immunomagnetic separation of HIV-infected cells, an HIV-1 drug screening assay is used to identify new anti-HV drug candidates' ability to block HIV-1 replication in vitro. A positive control is established by inoculating BTI's receptive CEMX174 cells in suspension with mixture of cultured laboratory isolates of HIV-1 (89.6). After 7 days of incubation, the cells are separated by centrifugation, washed and resuspended in PBS.

Anti-GP 120-FITC is introduced into the resuspended cells and incubated for 10 minutes. The cells are separated by centrifugation and washed. Anti-FITC Microbeads are used to separate the fluorescently labeled HIV-1-infected cells using positive selection columns. The HIV-infected cells are then quantified using standard flow cytometry.

The procedure for determining antiviral activity of new drug candidates is performed by adding serially diluted specimens of the candidate to the mixture of virus and CEMX174 cells and incubating for the same time as used for the positive control. Anti-GP 120-FITC and Anti FITC Microbeads are used in the same way as in the positive control to separate and enumerate the HIV-1-infected cells. The antiviral activity of each candidate is determined by noting the dose related differences from the positive control in the quantity of HIV-infected cells isolated after treatment and incubation of cells and virus with the drug candidate.

This same screening assay can be performed using other virally-infected cells, such as SIV or HTLV

The preceding examples can be repeated with similar success by substituting the generically or specifically described reactants and/or operating conditions of this invention for those used in the preceding examples.

The entire disclosure of all applications, patents and publications, cited above and below, and of parent applications Ser. No. 08/732,782 and 08/732,784, are hereby incorporated by reference in their entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of separating cells expressing a viral antigen, comprising:
a) contacting a target cell with a virus capable of infecting the cell, under conditions effective for achieving infection of the cell with the virus, to form a mixture;
b) adding to the mixture, a first binding partner specific for an antigen coded for by the virus which is expressed on the surface of the cell upon viral infection, under conditions effective for the first binding partner to bind to the viral antigen on the cell surface;
c) adding to the mixture resulting from b), a second binding partner specific for the first binding partner and attached to a magnetic bead, under conditions effective for the second binding partner to bind to the first binding partner, when the first binding partner is bound to the antigen expressed by the cell, to form a complex; and
d) separating target cells containing the complex, whereby said separation is achieved by a magnetic field.

2. A method of claim 1, further comprising adding to the target cell a sample antibody specific for the viral antigen.

3. A method of claim 2, further comprising measuring the number of target cells separated in d) in the presence and absence of the sample antibody

4. A method of claim 1, further comprising adding to the target cell a sample comprising an antibody specific for the viral antigen, whereby the amount of the second antibody is effective for interfering with the binding of the first binding partner to the viral antigen.

5. A method of claim 1, further comprising adding to the target cell a sample suspected of containing an antibody specific for the viral antigen.

6. A method of claim 5, further comprising measuring the number of target cells separated in d) in the presence and absence of the sample.

7. A method of claim 6, wherein the first binding partner is an antibody specific for the viral antigen.

8. A method of claim 6, wherein the second binding partner is an antibody specific for the first binding partner.

9. A method of claim 6, wherein the first binding partner is an antibody specific for the viral antigen, which antibody is labeled with a detectable label.

10. A method of claim 9, wherein the second binding partner is an antibody specific for the detectable label.

11. A method of claim 6, wherein the first binding partner is an antibody specific for the viral antigen, which antibody is labeled with a detectable label.

12. A method of claim 6, wherein the virus is HIV.

13. A method of claim 6, wherein the first binding partner is an antibody specific for the viral antigen gp120, which antibody is labeled with a detectable label; and the second binding partner is an antibody specific for the detectable label.

14. A method of claim 6, wherein the target cell is a T-cell line.

15. A method of claim 6, wherein the sample is a body fluid or blood.

16. A method of claim 6, wherein measurement of the number of target cells separated in d) in the presence and absence of the sample is accomplished by flow cytometry.

17. A method of claim 12, wherein the first binding partner is a receptor for the viral antigen.

18. A method of claim 16, wherein the first binding partner is a receptor for the viral antigen and is labeled with a detectable label; and the second binding partner is an antibody specific for the detectable label.

19. A method of claim 6, wherein the bead diameter is about 50-120 nm.

20. A method of claim 6, wherein the cell is contacted by at least about 100-1000 beads.

21. A method of identifying an agent which interferes with viral infection of a cell,
a) contacting a test cell with a virus capable of infecting the test cell, under conditions effective for achieving infection of the cell with the virus, to form a mixture;
b) adding to the resultant mixture formed in a), a test sample containing an agent suspected with interfering with viral infection of the test cell;
c) adding to the mixture of b), a first binding partner specific for an antigen coded for by the virus which is expressed on the surface of the test cell upon viral infection, under conditions effective for the binding partner to bind to the viral antigen on the cell surface;
d) adding to the resultant mixture formed in c), a second binding partner specific for the first binding partner and to a magnetic bead, under conditions effective for the second binding partner to bind to the first binding partner, when the latter is bound to the viral antigen expressed by the test cell, to form a complex;
e) separating test cells containing said complex, whereby said separation is achieved by a magnetic field; and
f) determining the number of cells infected with said virus in the presence and the absence of said test agent.

22. A magnetic bead having a surface coated by a cell-surface virus receptor for HIV.

23. A magnetic bead of claim 21, wherein the virus receptor is CD4.

24. A method of separating virus-infected cells from non-virus infected cells in a sample comprising,
combining (a) a first antibody recognizing a viral antigen on the surface of said cell and attached to a magnetic particle; (b) a second antibody recognizing said viral antigen on the surface of said cell and attached to a detectable label; and (c) a sample containing said virus-infected cells, to form a mixture;
incubating said mixture under conditions effective for binding of said antibodies to said viral antigen to form a complex, said complex comprising said first and second antibody bound to said virus-infected cell, and
moving said magnetic particle to a predetermined point on a reaction vessel holding said mixture, whereby said moving is accomplished by a magnetic field acting on said magnetic particle resulting in separating said virus-infected cells from non-virus infected cells, wherein said moving is accomplished without removing unbound antibody first and second antibody from said mixture.

25. A method of claim 24, further comprising detecting the label of said second antibody bound to said viral antigen on said virus-infected cell, wherein said first and second antibody recognize different epitopes of said viral antigen.

26. A method of separating cells infected with a virus, comprising:
a) contacting a target cell with a virus capable of infecting the cell, under conditions effective for achieving infection of the cell with the virus;
b) fixing and permeabilizing said cells;
c) adding to the fixed and permeabilized cells, a first binding partner specific for an antigen coded for by the virus, which viral antigen is ultimately expressed on the surface of the cell upon viral infection, under conditions effective for the first binding partner to bind to said viral antigen on the inside of said fixed and permeabilized cell;
d) adding to the result of c), a second binding partner specific for the first binding partner and attached to a magnetic bead, under conditions effective for the second binding partner to bind to the first binding partner, when the first binding partner is bound to the antigen expressed by the cell, to form a complex; and
e) separating target cells containing the complex, whereby said separation is achieved by a magnetic field.

27. A method of identifying an agent which interferes with viral infection of a cell, comprising:
a) contacting a test cell with a virus capable of infecting the test cell, under conditions effective for achieving infection of the cell with the virus, to form a mixture;
b) adding to the resultant mixture formed in a), a test agent suspected with interfering with viral infection of the test cell;
c) fixing and permeabilizing said cells;
d) adding a first binding partner specific for an antigen coded for by the virus, which viral antigen is expressed ultimately on the surface of the test cell upon viral infection, under conditions effective for the binding partner to bind to the viral antigen when said viral antigen is expressed in the interior of said cell;
e) adding to the resultant mixture formed in d), a second binding partner specific for the first binding partner and to a magnetic bead, under conditions effective for the second binding partner to bind to the first binding partner, when the latter is bound to the viral antigen expressed by the test cell, to form a complex;
f) separating test cells containing said complex, whereby said separation is achieved by a magnetic field; and
g) determining whether the test sample changes the number of test cells containing the complex when compared to the process performed in the absence of said agent.

28. A method claim 27, where said test agent is added to cells prior to simultaneous to contacting cell with said test agent.

29. A method of separating cells expressing a cell-surface viral antigen, comprising:
a) combining an effective amount of an anti-cell-surface viral antigen antibody attached to a detectable label, an effective amount of an antibody specific-for said detectable label, and an aqueous sample containing viral-infected cells displaying said cell-surface viral antigen, to form a mixture, wherein said antibody specific-for said detectable label is attached to a magnetic particle;
b) incubating said mixture under conditions effective for binding of said anti-cell surface viral antibody to said cell-surface viral antigen, and, for binding of said antibody specific-for said detectable label to said detectable label attached to said anti-cell surface viral antibody, to form a complex, wherein said anti-viral antibody is bound to said cell-surface antigen displayed on a viral-infected cell; and
c) separating said complex, comprising said cells expressing said cell-surface viral antigen and magnetic particles, by applying a magnetic field to said mixture, whereby said complex is retained by said magnetic field.

30. A method of claim 29, wherein viral-infected cells are infected with HIV.

31. A method of claim 29, wherein said cell-surface viral antigen is an envelope glycoprotein for HIV.

32. A method of claim 29, wherein the envelope glycoprotein is gp120 or gp41.

33. A method of claim 29, wherein said anti-cell surface viral antibody is a polyclonal antibody specific for HIV envelope glycoprotein and said viral-infected cells are infected with HIV.

34. A method of claim 29, wherein said detectable label is FITC, TRITC, or R-phycoerthryin.

35. A method of claim 29, further comprising counting said magnetically-separated cells by flow cytometry.

36. A method of claim 29, wherein said magnetic particles are about 10-150 nm in diameter.
A method of separating cells expressing a cell-surface viral antigen, comprising:
a) combining an effective amount of an anti-cell-surface viral antigen antibody attached to a magnetic particle and an aqueous sample containing viral-infected cells displaying said cell-surface viral antigen, to form a mixture;
b) incubating said mixture under conditions effective for binding of said anti-cell surface viral antibody to said cell-surface viral antigen displayed on said viral-infected cells, to form a complex; and
c) separating said complex comprising said cells expressing said cell-surface viral antigen and magnetic particles by applying a magnetic field to said mixture, whereby said complex is retained by said magnetic field.
